(19) United States
(12) Patent Application Publication
(10) Pub. No.: US 2018/0132953 A1
(43) Pub. Date: May 17, 2018

(54) TELESURGICAL SYSTEM WITH INTRINSIC HAPTIC FEEDBACK BY DYNAMIC CHARACTERISTIC LINE ADAPTATION FOR GRIPPING FORCE AND END EFFECTOR COORDINATES

(71) Applicant: TECHNISCHE UNIVERSITÄT DARMSTADT, Darmstadt (DE)

(72) Inventors: Carsten Neupert, Pfungstadt (DE); Christian Hatzfeld, Schwalbach (DE); Sebastian Matich, Pfungstadt (DE)

(73) Assignee: TECHNISCHE UNIVERSITÄT DARMSTADT, Darmstadt (DE)

(21) Appl. No.: 15/544,353

(22) PCT Filed: Jan. 18, 2016
(86) PCT No.: PCT/EP2016/050901

§ 371 Date: Dec. 1, 2017

(30) Foreign Application Priority Data
Jan. 19, 2015 (DE) 10 2015 100 694.4

(51) Int. Cl. A61B 34/35; A61B 34/00; A61B 90/00; B25J 9/16; B25J 3/00; B25J 13/08; B25J 19/02; B25J 13/02; G01L 5/22

(52) U.S. Cl. CPC A61B 34/35; A61B 34/74; A61B 34/76; A61B 34/77; A61B 90/361; B25J 9/1689; A61B 2034/301; B25J 13/085; B25J 19/023; B25J 9/161; B25J 13/025; G01L 5/226; B25J 3/00

(57) ABSTRACT

A teleoperation system is provided, having a slave having a drive unit which drives a gripping end effector, wherein a kinematic coordinated end effector and a gripping force f effector can be determined with a camera which is preferably integrated in the slave and which is aligned with the end effector; a master, which is remote from the slave, with at least one operating unit on which a user can exert a gripping head F_{G}, the gripping force being transmitted to the slave, and a visual user interface representing the image of the camera; and where F_{G} is linearly dependent on the kinematic coordinate and the F_{effector}.

The invention relates to a teleoperation system based on a master-slave structure.

### BACKGROUND OF THE INVENTION

Background of the invention is the development of a teleoperation system for a medical application. The teleoperation system is intended to provide haptic feedback for the representation of interaction forces, preferably between an end effector and the surrounding tissue.

For use in surgery, telemanipulation systems, in the following also called teleoperation systems, exist, which can be referred to as a remote-controlled system. In particular, the limited integration of haptics and the implementation as a pure telemanipulation system are limitations for a wider use in surgical environment. Thanks to the use of lightweight robotics with comprehensive integrated force/torque sensors, completely novel approaches for surgical interventions are possible. The integration of haptic processes in the context of therapeutic and diagnostic concepts in medicine represents the next stage for an intuitive human-machine interface. Also, the extension of pure telemanipulation to a teleoperation with the integration of autonomous partial procedures also relieves the doctor of concentration-reducing routines.

The term haptics originates from the Greek. It means “tangible” or “suitable for touching”. In principle, therefore, all media offer the possibility of haptic perception. They feel in a certain way. A table surface can be smooth or rough. It is therefore a perception, which occurs primarily by the fingers of the hand.

With the pseudo-haptic feedback, the user is given a haptic impression via additional visual information. For example, the information on a screen gives the user the impression that a haptic feedback is present, which is actually not the case or only minimal.

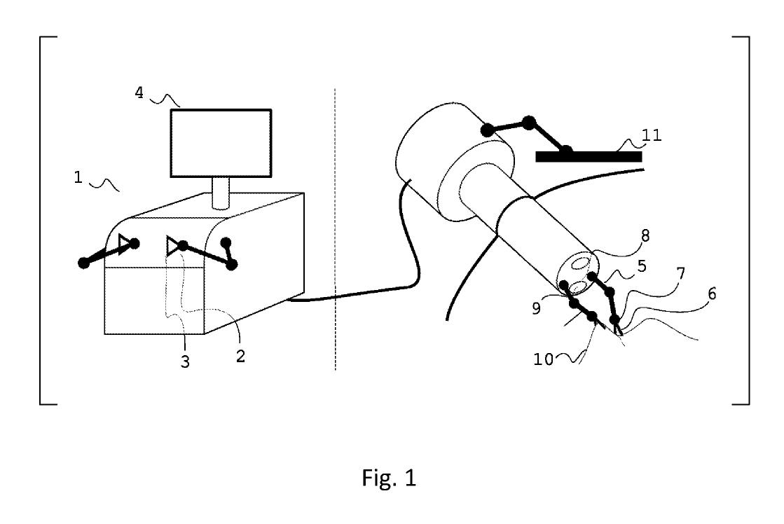

In a teleoperation system based on a master-slave structure, the master, at whom the doctor sits, comprises a control unit. The control unit preferably includes two control means for the left and right hand (left, right). The doctor interacts with the control. The operational area is presented to the user by a visual user interface, such as a screen. The doctor should see on the screen only the operation area, or the end effector. For an intuitive operation it can be an advantage if one does not see his own hands during the teleoperation. In this case, it is of advantage, especially in the case of pseudo-tactics, that one cannot see his own fingers, since the irritation due to the missing or deviation of the finger from the expectation does not occur. FIG. 1 shows a corresponding system.

The slave, also referred to as a single-port robot, consists of a drive unit. Two parallel kinematic manipulators (left/right) are controlled by push rods with the movements generated in the drive units. At the top of each manipulator is the Tool Center Point (TCP), which is used to hold surgical tools (end effectors), and can be positioned in the situs, for example. The slave has one or more drives, which are arranged distally as far as possible from the end effector in the extension of the push rods of the parallel kinematic manipulator in order not to have any negative effect during sterility. The slave further comprises a camera, illuminants, and preferably a working channel. The two systems are electrically connected in the control computer.

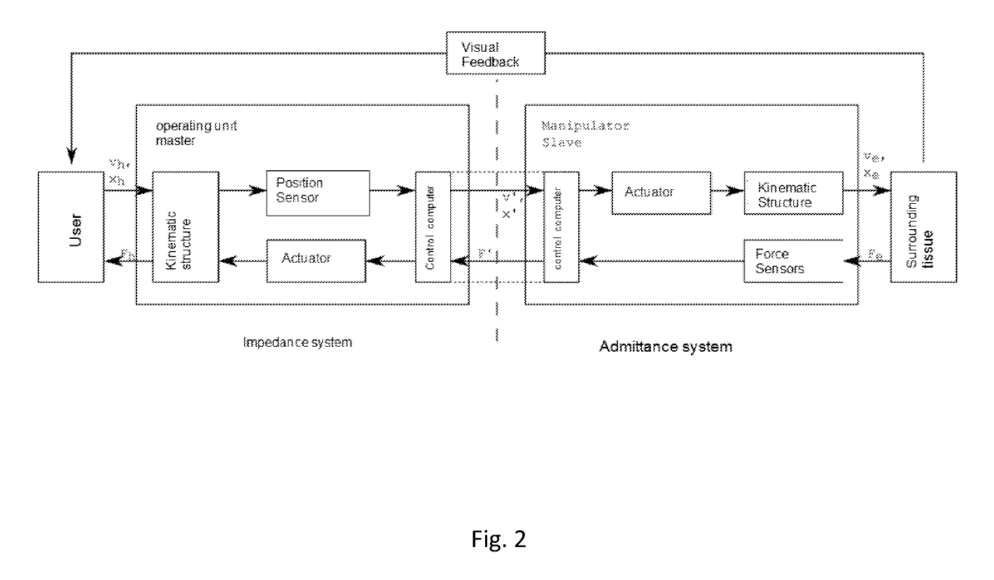

FIG. 2 shows the system structure of a conventional system. This system consists of an impedance system, referred to as a master, and an admittance system, which is also called slave. The master system comprises a man-machine interface, which generally consists of a screen and corresponding input means. The user sends position instructions to the slave via a kinematic structure. Via corresponding position sensor systems, these are passed to a control unit, which then drives one or more actuators, which are located in the slave. The actuator in turn controls a kinematic structure, which then has access to the environment or the tissue. By one or more force sensors, the feedback is given by the slave via the control unit, which in turn drives one or more actuators in the master unit which exert an influence on the kinematic structure, which generates a haptic feedback to the user. These sensors and actuators give the user an indirect feedback.

### OVERVIEW OF THE INVENTION

The object of the invention is now to provide a realistic pseudo-tactical feedback without integrating a (further) actuator in the user interface for the active generation of the haptic feedback. Likewise, a sophisticated force sensor system in the end effector can be dispensed with by this method.

The object of the invention is to generate a pseudohaptic feedback in the control unit of a teleoperation system. In this case, compared to the current state of the art, an actuator in the user interface is dispensed with and the measuring technology expenditure in the end effector is reduced. The pseudohaptic feedback is generated by utilizing the visual feedback during the application and the processing of different sensory impressions to a consistent perception by the user.

Specifically, it is a teleoperation system comprising:
A slave, which has a drive unit, which drives a gripping end effector, wherein a kinematic coordinate of the end effector and a gripping force F_{effector} can be determined. The kinematic coordinate is, for example, a closing angle for rotational freedom degree or a travel path for translatory freedom degree of the end effector. In this application, the closing angle Phi is used for the above-described class of end effectors, so as to include the kinematic coordinates. This is the case, in particular, when the end effector is not closed in a shear-like manner or rotationally via a joint, but by a linear travel. The drive unit is also called an actuator and can be a motor or a plurality of motors with and/or without a transmission or clutch. This motor is arranged in a slave housing as far away as possible from the tissue in order to prevent impurities. The motor drives the end effector, and in particular its gripper. It should be noted that there are also other motors in order to be able to implement further functions of the end effector or further end effectors. Also, there may be other motors to perform multi-dimensional movements.
Another component of the teleoperation system is a camera which is preferably integrated in the slave and which is aligned with the end effector. The camera can also be attached to another device, but it should provide a view of the end effector and its gripper. The camera allows visual feedback. In a further embodiment, an additional digital representation of the current end effector coordinate can be superimposed on the camera image. (Angle indication, lines which move towards each other, a stylized gripper that moves, color shifts, distances, deflections). It is also possible to display the force acting on the end effector on the display. This would lead to an “augmented reality”.
Another component of the teleoperation system is a master which is connected to the remote slave. The connection can be via radio or cable. The master has at least one control unit, on which a user can apply a gripping head F_{G}. In general, the control unit contains two operating devices, which are used for the right and left hand. With these control units, movements can be performed, which can generally be executed in several dimensions. Gripping with the end effector with the gripping force F_{G} is generally effected by a pressure with the fingers on a pressure region, which is formed in the control means of the control unit, whereby the gripping force or information of the gripping force is transmitted to the slave. Furthermore, the master comprises a visual user interface which represents the image of the camera and thus allows feedback. The information on the gripping force is first transmitted to the control computer. The control computer converts the gripping force into an opening angle specification for the end effector, depending on the given mathematical relationship, and sends this to the slave. In the case of the device, it must be noted that F_{G} is linearly dependent on the closing angle/a kinematic coordinate and the F_{effector}. That the closing angle is determined by the gripping force on the control means and by the force determined at the end effector. The larger the two forces, the smaller the angle between the two grippers of the end effector. In particular, the larger the ratio between the two, the greater the closing angle. For the pseudo-haptic perception of the interaction force acting at the end effector, there is thus no need in the control unit for an active actuator component which produces a feedback.

In a further embodiment, the F_{effector} is determined by one or more of the following approaches:
derivation of the force from control variables and/or control parameters, as well as model assumptions of the drive unit in the slave,
Measurement of the current in the drive unit,
Measurement of the force in a kinematic structure between the end effector and the drive unit.

These may be e.g. Struts or guide rods or joints.
Structure-integrated measurement in components of the slave, deriving the forces of the end effector. These may be e.g. bearing or housing parts.
By structure-integrated force sensors in a parallel kinematic manipulator, which measure forces and moments in the struts and/or the bearing reaction forces in the joints of the parallel kinematic structure. These may be e.g. Uniaxially in the struts or at one point in a multidimensional manner.
Force/torque sensors on the drive unit. This can be done before and after the transmission unit/gear box.
measurement of the force directly between the end effector and the surrounding tissue by means of sensors which are applied punctionally or laminar to the sections of the end effector.

In a possible embodiment, the control unit is, in particular the control means, as rigid as possible and has only the flexibility necessary for the gripping force detection. The user interface should be rigid in order to obtain the following advantages (in the pseudo-freedom degree, do not allow any deflection).
No loss of dynamics when transmitting active haptic feedback from other degrees of freedom.
Very good connection of “highly dynamic” feedback in the rigid control unit.
No movement of the fingers and thus loss of adhesion of the user on the control means.

However, the control means can also be designed with a constant resilience and thus for a defined deflection. This results in better (more realistic) results for the degree of freedom of pseudohaptic feedback, but loses the advantages described above for the overall system.

In a further embodiment, the gripping force F_{G} is determined by deriving the interaction force between the control means and the user by one or more of the following methods:
Simple force measurement between the fingers
Differential force measurement between the fingers.

This preserves the independence between gripping force (pseudohaptic feedback) and possible active haptic feedback of other degrees of freedom. The differential force measurement is achieved by measuring the gripping force of the thumb and index finger separately from each other. (In practice, the smaller and possibly the larger of the two measured values of the gripping force is presumed). If the differential force of the thumb and index finger is measured separately, the parasitic forces can be calculated by external feedback and as a result on has only the really effective force between thumb and pointing fingers. Differential force measurement thus makes it possible to measure the force independently of disturbances. Disturbances are, in this connection, additional forces which, for example, integrate spatial feedback.
From deflection, deformation of a non-rigid operating device This essentially results in the fact that one of the following dependencies can apply to the teleoperation system, where

F_{G}=Kinematic coordinate*F_{effector}

### Or

F_{G}=Kinematic coordinate+F_{effector}

or

F_{G}=Kinematic coordinate*(F_{effector}+Fₘᵢₙ)+F_{G}_{_}_{offset}

Where Fₘᵢₙ is the force to initially move the effector, and F_{G}_{_}_{offset} is the force to allow the sensor to respond in the control unit. Other dependencies, in particular linear, are also conceivable. It should be noted that the formulas should only represent the basic/general dependency. Alternate parameters can be considered, which are not yet included here. These include scaling factors of the individual forces as well as scaling factors for adapting the units in the equation and adapting them to arbitrary kinematic coordinates. The kinematic coordinate can be represented, inter alia, by the closing angle of an end effector.

For the purposes of the invention, all mathematical relationships between the gripping force F_{G} and the kinematic coordinate of the end effector fulfill their purpose, in which an increase in the acting end effector interaction force leads to a greater necessary gripping force of the user in order to bring about a further increase in the kinematic coordinate.

The selected relationship as well as the scaling factors should be selected depending on the environment manipulated by the end effector.

Pseudohaptic feedback works up to a frequency of approx. 10 Hz. This barrier results from the ability of humans to self-consciously generate forces and movements up to this frequency. (DIN EN ISO 9241 910). This serves mainly to the haptically kinesthetic senses.

Tactile haptic feedback can be output for frequencies that go beyond this. For this purpose, an actuator system can be used in the user interface, which couples a direct or non-direct haptic feedback to the user. (Frequency range approx. 50 Hz-1000 Hz according to DIN EN ISO 9241 910). Thus, information regarding material selectivity, surface structures, etc. can be presented.

In a further embodiment, a unit for generating a tactile haptic feedback is used on the control unit or control means, whereby a signal, which is sent to the unit for generating tactile haptic feedback, is detected by a sensor in the slave, the spectral components of this signal are preferably in the range from 50 to 1000 Hz.

The output of the previously described tactile haptic feedback can be effected by:

1. Force output by inertial mass motors
2. Eccentric motors
3. Piezo actuators—Direct between the control unit and the control unit
4. Piezo actuators—Intermediate between the base of the control unit and the fingers
5. Piezo actuators for generating surface waves at any location of the control means
Thus, on the one hand, controlled force variables, or accelerations, can be represented.

In a further embodiment, the above-mentioned elements are formed in such a way that the acting force direction of the unit for generating tactile haptic feedback exert no or only minimal forces in the direction of the gripping force F_{G} in order to reduce control instability in the system.

In one embodiment, when such a feedback is introduced, an attempt is made to remove the forces and deflections exerted from the actually acting force direction in order to open the control circuit and thus reduce control-technical instabilities in the system. In addition, the position pre-selection signals can be “notch-filtered” (narrow-band filters or notch filters) depending on the output “high-frequency” tactile output values in order to obtain the control-related stability in the haptic system. By using a notch filter a narrow band elimination of a certain frequency is possible. This can be adapted adaptively to the frequency of the tactile feedback. In one embodiment, the position presetting signals may also be passed through a low-pass filter with a cutoff frequency below the typical tactile feedback frequencies, e.g. 40 Hz, so as to separate the frequency ranges of the channels from each other.

In one embodiment, the sensor in the slave is an acceleration sensor. Alternatively, encoder signals of the actuators can be used. High-frequency signals can also be derived from force sensors which have already been described. One could also imagine using “surface acoustic wave” (SAW) sensors to detect surface vibrations in the kinematic components or at the end effector.

Another part of the invention is the construction of the slave for a teleoperation system, e.g. as described above. The slave can of course also be used for other systems and is not limited to a teleoperation system and vice versa. Components can also be used in other combinations.

The slave includes:

At least three pushing rods arranged as tripod, each having two active degrees of freedom in the form of translation and rotation, and each being driven by means of a drive into the degrees of freedom. More pushing rods can also be possible, their arrangement can also be different;

An end effector which is connected to the push rods via kinematic chains, the kinematic chains being designed in such a way that the end effector can be aligned and opened and closed in three dimensions, by translation or rotation of the push rods.

In one possible embodiment, there is a kinematic chain, which is designed as a main chain, the rotation of which leads to a rotation of the end effector and its displacement leads to a displacement of the end effector.

In addition, there are two chains which are formed as side chains whose displacement results in a displacement of the end effector and whose rotation leads to an opening or closing or angling of the end effector.

In one possible embodiment, the rotations of the secondary chains are converted into a linear movement via a spindle and a carriage, which opens or closes the angled end effector.

The kinematic main chain preferably has four degrees of freedom and/or the kinematic secondary chain preferably has six degrees of freedom.

The secondary chain is preferably connected to the main chain or its pushing rod via swivel joints, wherein the swivel joints are designed as U-shaped tensioning elements.

In sum, more favorable, more robust and more easily sterilizable systems can be developed with the invention. The use of pseudo-haptic feedback in teleoperation systems has advantages over conventional haptic teleoperation systems in terms of regulatory stability.

### BRIEF DESCRIPTION

FIG. 1 shows the structure of an exemplary teleoperation system based on a master-slave structure;

FIG. 2 shows the system structure of a conventional teleoperation system;

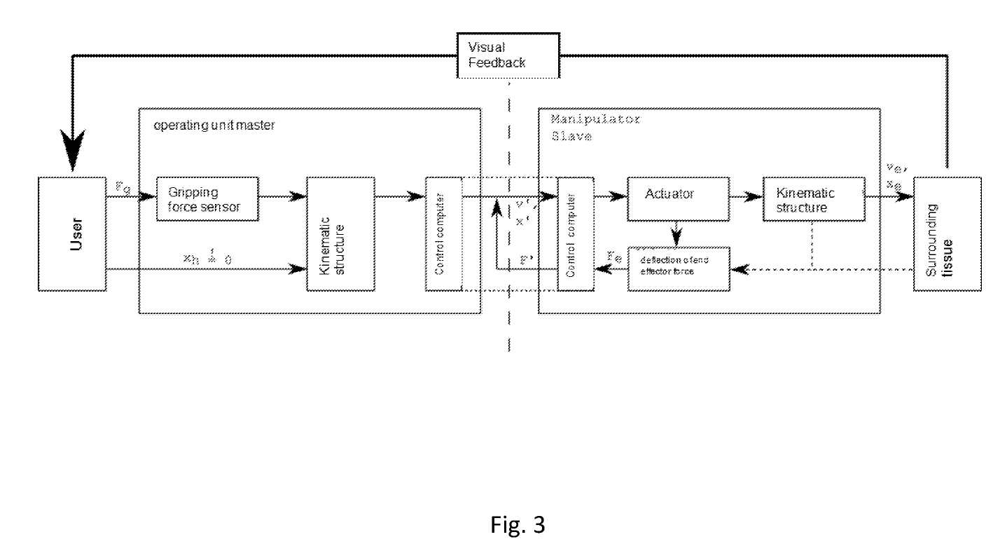

FIG. 3 shows the system structure of a “pseudohaptic” system;

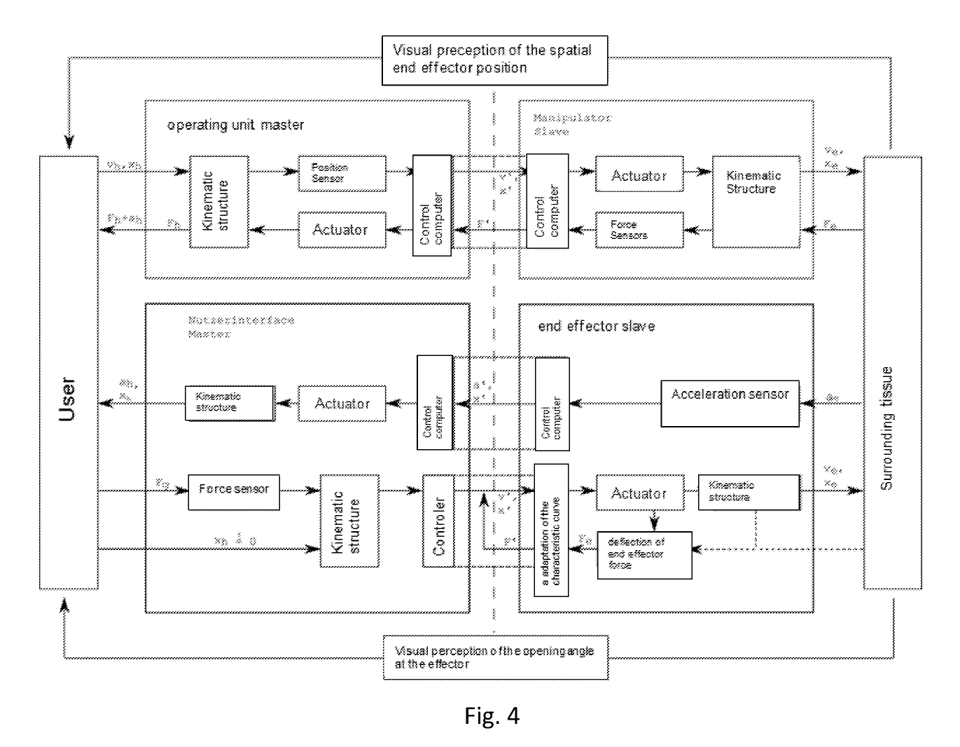

FIG. 4 shows the system structure of a combined teleoperating system impedance admittance structure as well as an additional pseudohaptic degree of freedom and a structure for superposition of high-frequency haptic feedback;

FIG. 5 shows an end effector with different positions of the gripping arms;

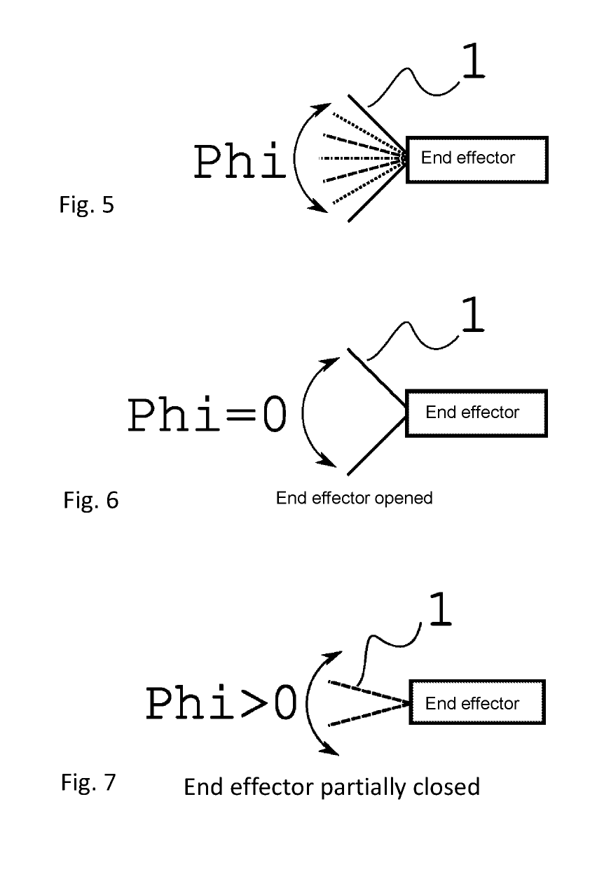

FIG. 6 shows an end effector with completely opened gripper arms;

FIG. 7 shows an end effector with partially closed gripping arms;

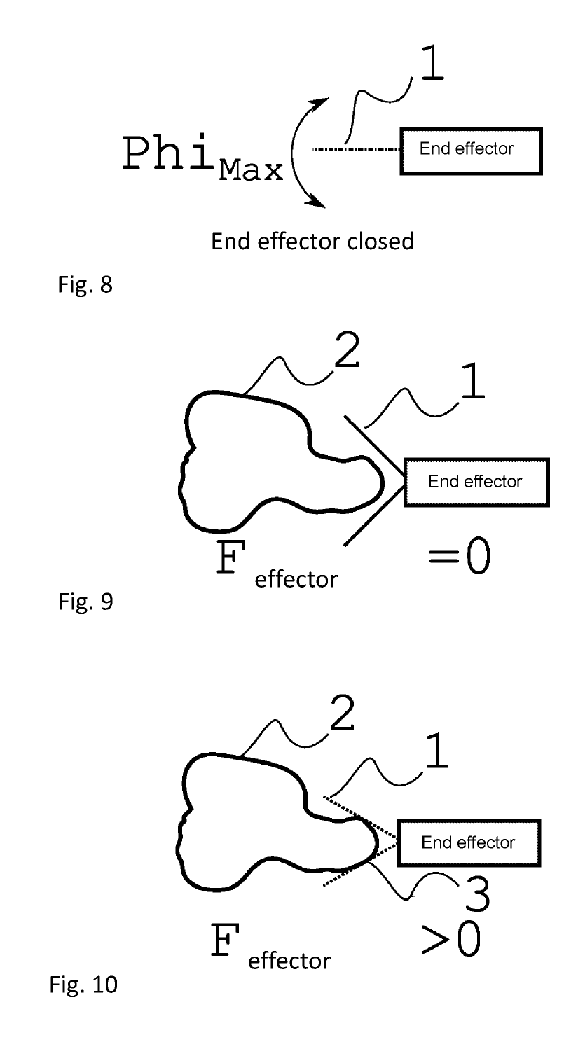

FIG. 8 shows an end effector with completely closed gripper arms;

FIG. 9 shows an end effector without force action between the gripper arms, since no tissue contact is yet present;

FIG. 10 shows an end effector with an effective end effector gripping force during tissue contact;

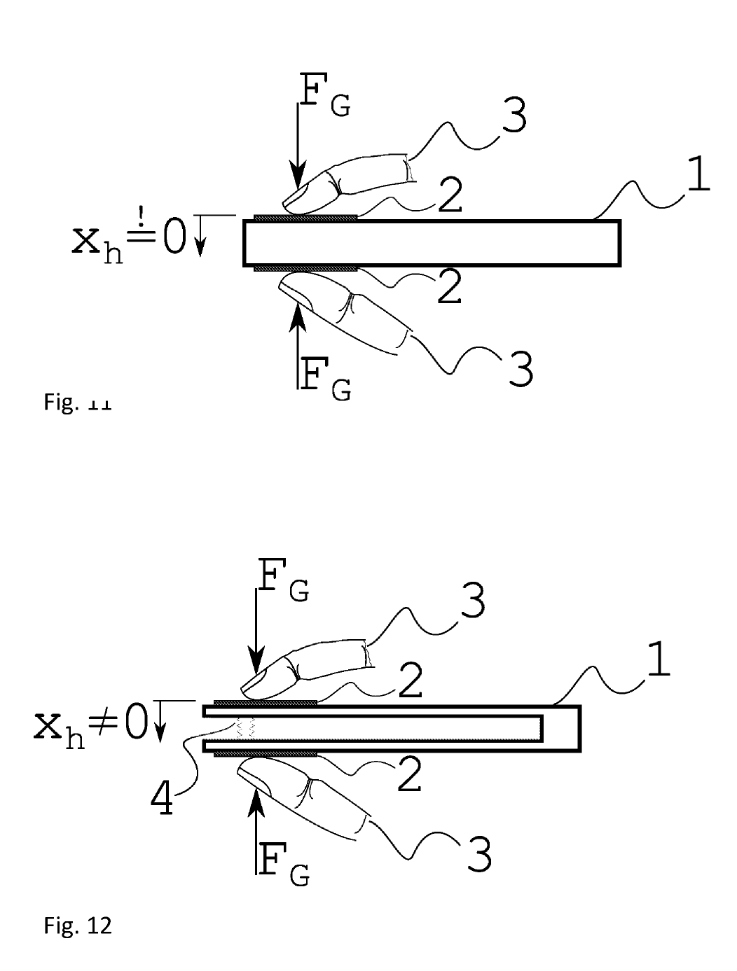

FIG. 11 shows a rigidly designed control means in the master as well as the direction of the gripping force under engagement of the user;

FIG. 12 shows a control means which is designed with a defined resilience as well as the direction of the gripping force under engagement of the user;

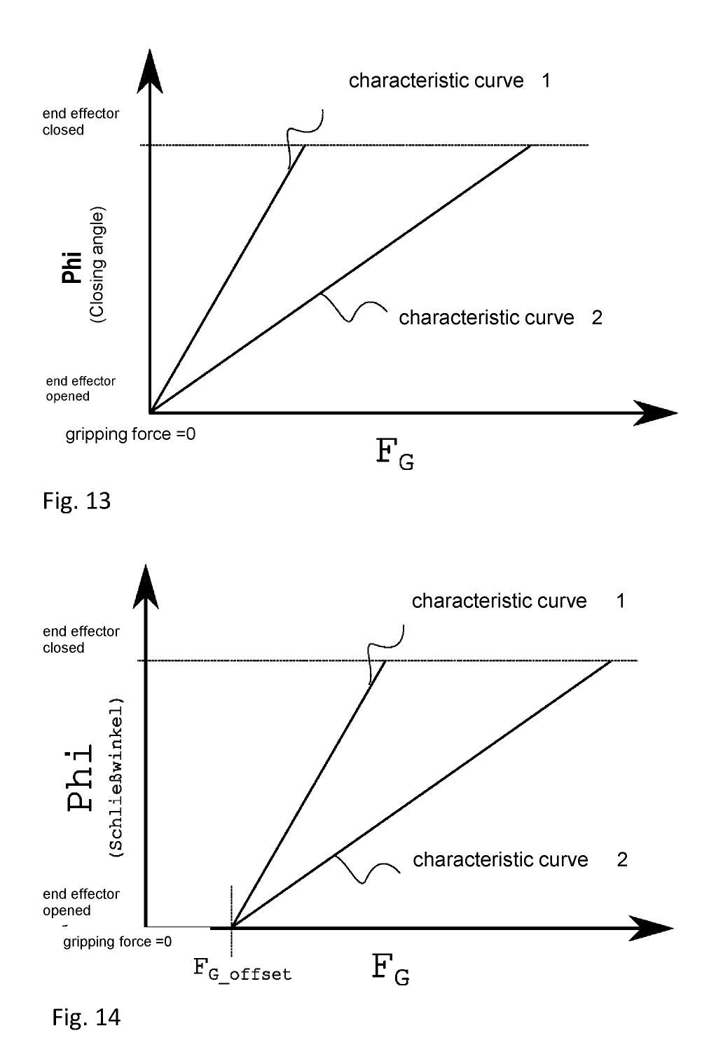

FIG. 13 shows the relationship between the gripping force and the closing angle without influencing the coupling characteristic by the effective end effector force. Characteristic curve 1 and characteristic curve 2 differ by the predetermined force Fₘᵢₙ;

FIG. 14 shows, in contrast to FIG. 13, the relationship between gripping force and closing angle starting from an optionally applicable offset of the gripping force;

FIG. 15 shows the haptically perceptible gripping force difference in the case of visually perceived equal end effector closing angle by variation of the coupling characteristic between gripping force and closing angle;

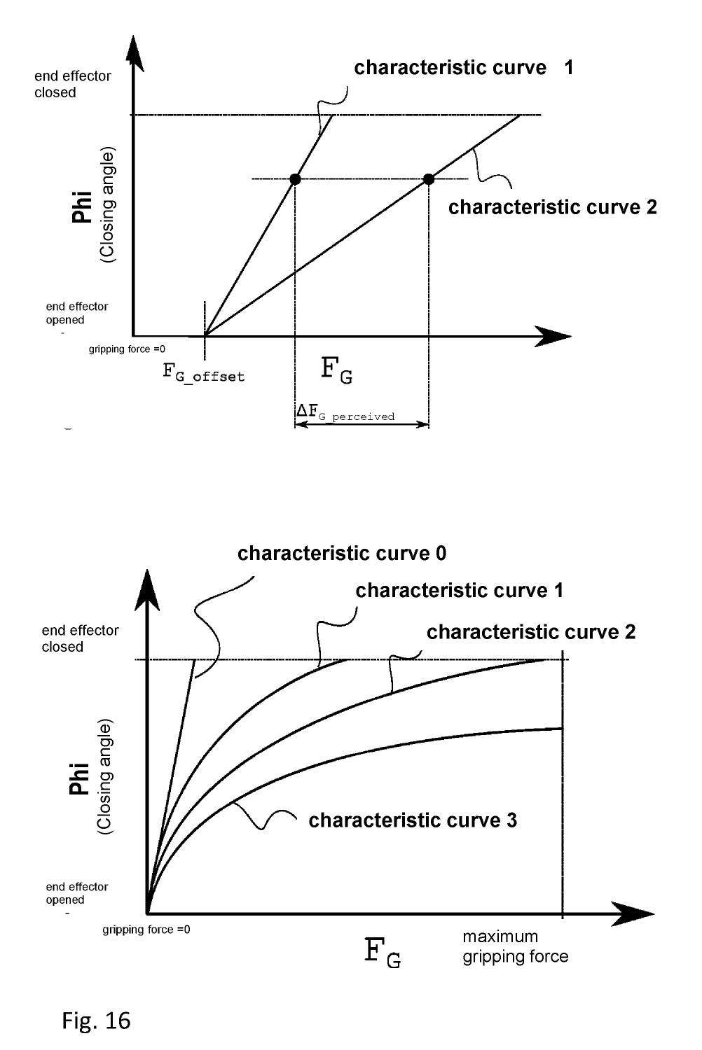

FIG. 16 shows an exemplary characteristic curve with the influence of different end effector gripping forces based on the multiplicative evaluation of the relationship between gripping force and closing angle with the acting end effector gripping force.

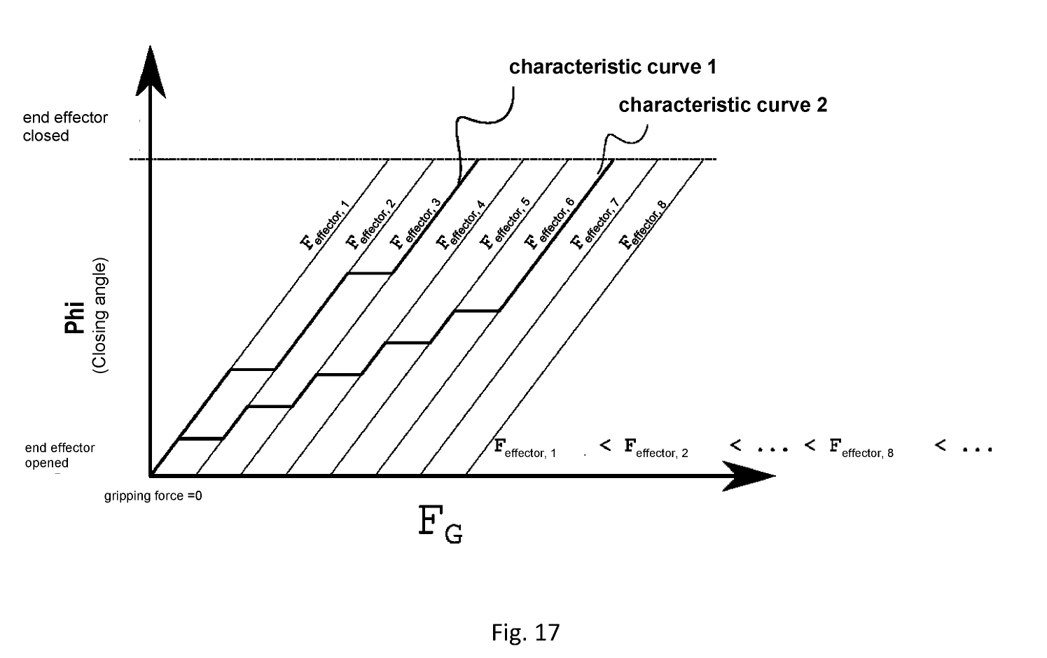

FIG. 17 shows, in contrast to FIG. 16, the characteristic curve profile with the influence of different end effector gripping forces on the basis of the additive assessment of the relationship between gripping force and closing angle with the acting end effector gripping force.

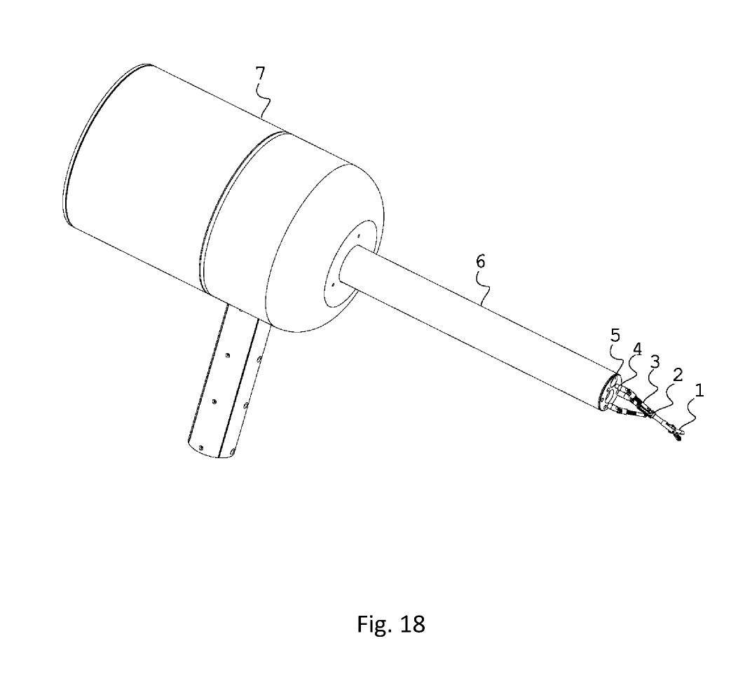

FIG. 18 shows the embodiment of the slave consisting of end effector 1, TCP 2, parallel kinematic mechanism 3, push rods 4, camera channel 5, shaft 6 and drive unit 7;

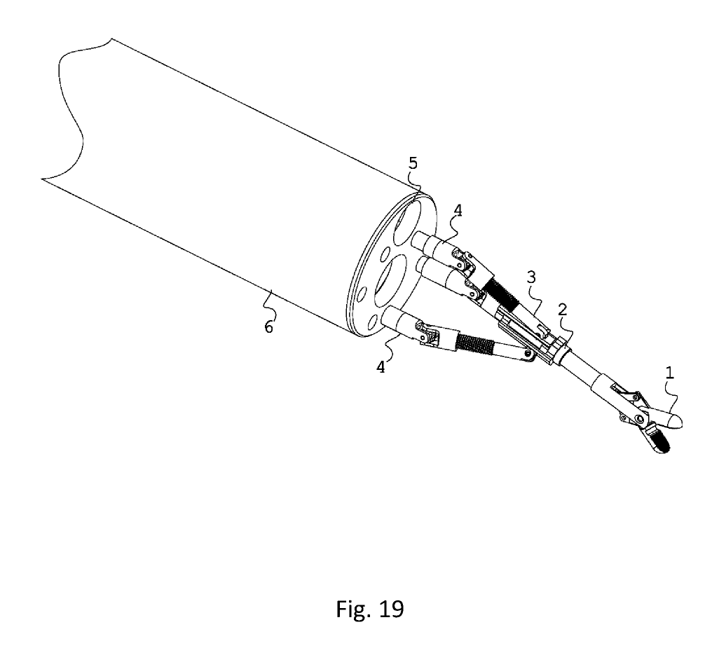

FIG. 19 shows an enlargement of the embodiment in FIG. 18 with end effector 1, TCP 2, parallel kinematic mechanism 3, push rods 4, camera channel 5, shaft 6;

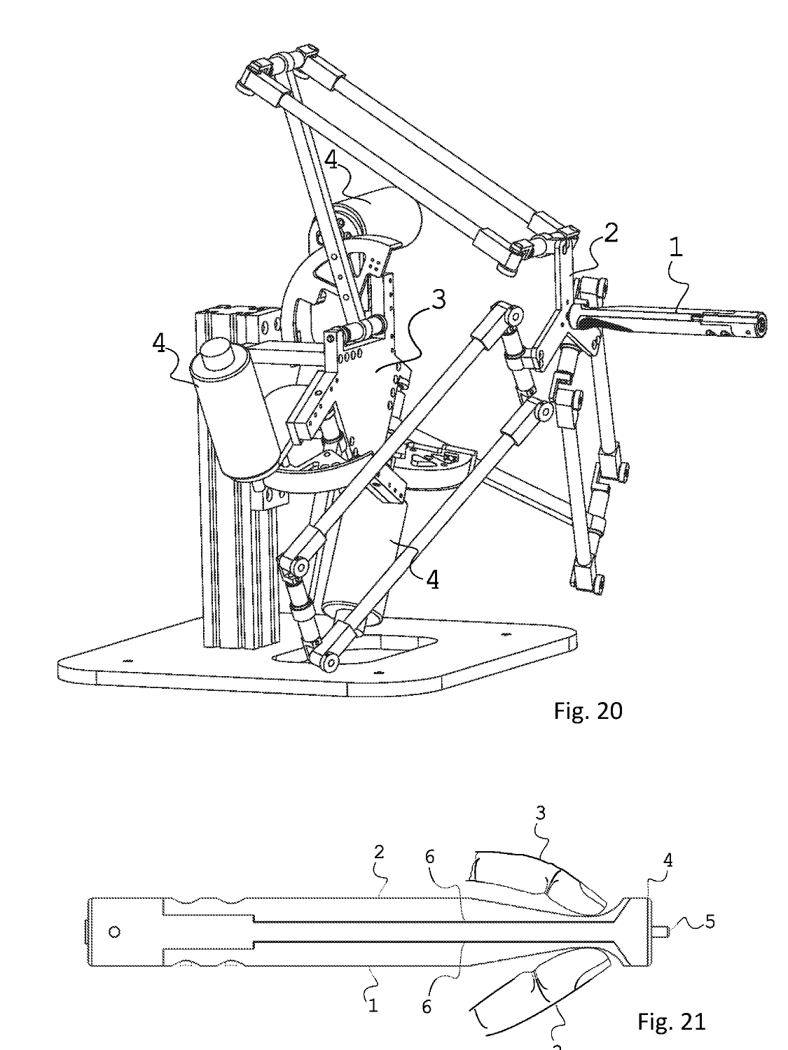

FIG. 20 shows the embodiment of an control means with control means 1, TCP of the control unit 2, base 3, drives of the control means 4;

FIG. 21 shows the embodiment of a rigid control means with gripping elements 1, 2, fingers 3, base 4 of the control means, fastening element 5 for fastening to the TCP of the control means, force sensor elements 6 between grip elements and base of the control means;

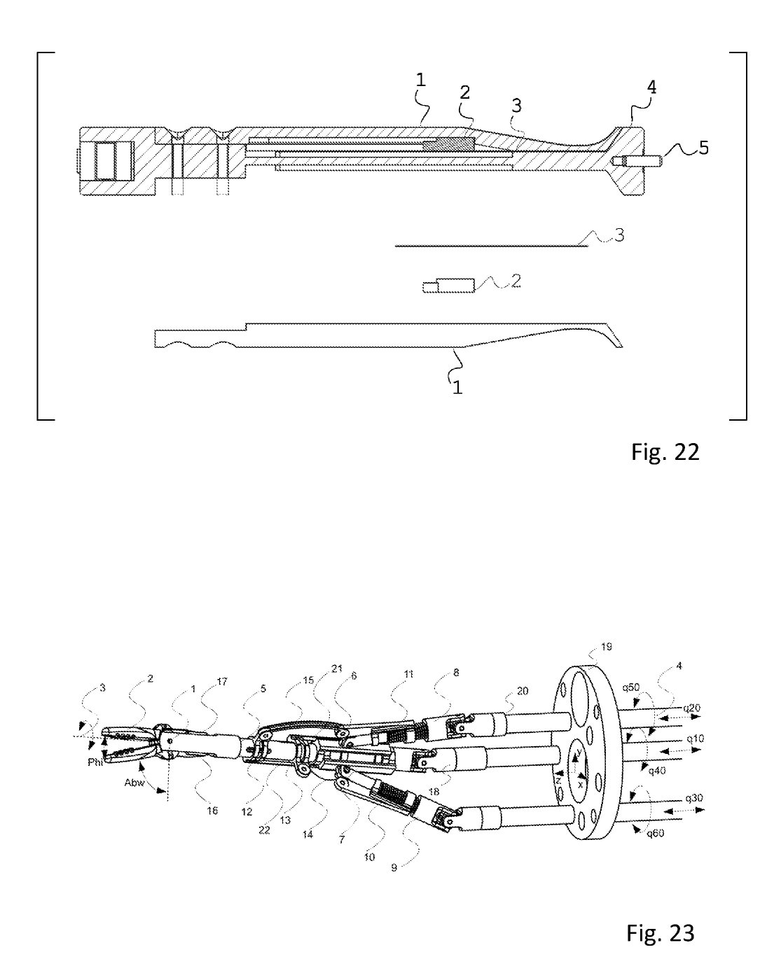

FIG. 22 shows a sectional as well as the exploded drawing of the embodiment of an control means with grip elements 1, drives for tactile feedback 2, force sensor elements 3, base of the control means 4, fastening element for TCP of the control means;

FIG. 23 shows a detailed construction of the slave.

### DESCRIPTION OF THE EMBODIMENT

The invention is described below with reference to a teleoperation system for minimally invasive surgery, which is not to be understood as limiting. This transmits control information from the user to an intracorporal manipulator and represents the interaction forces between the end effector of the intracorporal manipulator and tissue as haptic and pseudo-haptic feedback on the control unit.

FIG. 1 shows the structure of an exemplary teleoperation system based on a master-slave structure with master 1, control unit 2, control means 3, visual user interface (screen 4), parallel kinematic mechanism 5, end effector 6, tool center point 7, working channel 8, camera channel 9, Slave 10, operating table 11.

The slave is shown in FIG. 1. It consists of a parallel kinematic mechanism to which TCP an end effector is mounted. The position of the TCP and thus the position of the end effector can be adjusted by the defined longitudinal displacement of the push rods. The individual push rods are moved by separate actuators in the drive unit. The slave's shaft has a channel for a camera and a working channel. The end effector consists of two gripping arms, between which the end effector gripping force (F_{effector}) acts. The closing angle (Phi) is the angle between the two gripping arms of the effector. Both the acting force (F_{effector}) and the closing angle (Phi) are thus determined. In addition to the force between the end effector grippers (F_{effector}), the interaction forces between the end effector and the environment are derived. The slave is connected to the master by means of a corresponding cable.

FIGS. 2 and 3 show in comparison the difference of a conventional system to the system of the present invention. It can be seen here that feedback via actuators is not given in the present invention. FIG. 4 shows the system structure of a combined teleoperating system, impedance admittance architecture as well as an additional pseudohaptic degree of freedom and a structure for superposition of high-frequency haptic feedback. The systems of FIGS. 2 and 3 have been combined together here.

The master consists of two control units according to FIG. 20 for the left hand and the right hand. These control units have a control means according to FIGS. 11, 12, 20, 21, 22. The user operates with the control means via the control unit. The control means is connected to the control unit at the TCP of the kinematics of the control unit. Control inputs for the slave are entered into the system by means of user input into the control means and thus into the control unit. By means of actuators mounted in the base of the control means, haptic feedback can be generated with regard to the interaction forces measured at the slave between the end effector and the environment and can be output to the user via the control means.

FIG. 18 shows an embodiment of the slave consisting of end effector 1, TCP 2, parallel kinematic mechanism 3, push rods 4, camera channel 5, shaft 6 and a drive unit 7.

FIG. 19 shows an enlargement of the embodiment in FIG. 18 with end effector 1, TCP 2, parallel kinematic mechanism 3, push rods 4, camera channel 5, shaft 6.

FIG. 20 shows the embodiment of a control mean with control means 1, TCP of the control unit 2, base 3 and the drive of the control unit 4.

FIG. 21 shows the embodiment of a rigid control means with gripping elements 1, 2, fingers 3, base 4 of the control means, fastening element for fastening 5 on the TCP of the control means on the control unit, force sensor elements 6 between grip elements and base of the control means.

The gripping force of the user is used as the control variable for the closing angle phi of an intracorporal end effector (see, for example, FIGS. 6 to 17) instead of a position measurement of movable elements of the control means. For this purpose, a force sensor system is used in the control means for detecting the gripping force (for example, FIGS. 12 and 22). By adjusting the required gripping force F_{G, max} for the complete closing of the end effector, the behavior of the end effector can be influenced in the form of a (linear) characteristic phi (F_{G}) and modified in a manner adapted to the situation (FIGS. 13-17). A haptic sense impression is thereby produced by the correlation of gripping force itself introduced into the user interface and the visually perceived closing angle of the end effector. See FIGS. 7-9.

In order to generate the haptic feedback, no actuator is necessary in this case since the user generates the force necessary for a haptic sense impression by virtue of its gripping force. A necessary prerequisite is a direct view of the end effector by the user. The fundamental function of this “pseudohaptic feedback” is known from the realm of virtual reality.

The force F_{G} or also F_{grip} is determined on the control means as shown in FIGS. 11,12.

In order to ensure haptic feedback of a material in the end effector/gripper, the characteristic curve (FIG. 13-17), which represents the relationship between gripping force at the user interface and the closing angle of the end effector, can be varied (see FIGS. 5-10).

This takes place as a function of the force required for closing or actuating the end effector. This corresponds to the interaction force F_{effector} due to the adjusting force balance.

The variation of the characteristic curve phi (FG) is thereby possible by adding the measured output end effector force

phi′=phi (F_{G}+F_{effector}) as well as by multiplying the measured end effector force phi′=phi (F_{G}F_{effector}). The two cases describe a differently strong weighting of the respectively effective end effector force (F_{effector}). In both cases, the necessary gripping force, which is necessary to achieve a certain closing angle phi, changes. In connection with the visual feedback on the opening of the gripper, an impression is thus obtained for the user of the material at the end effector, since the interaction force F_{effector} is inter alia material-dependent.

FIG. 16 shows an exemplary characteristic curve with the influence of different acting end effector gripping forces on the basis of the multiplicative evaluation of the relationship between gripping force and closing angle with the acting end effector gripping force. Characteristic curve 0 shows the course of the coupling characteristic curve without an effective end effector gripping force. Characteristic curves 1 and 2 show the course of the coupling characteristic curve for gripped materials of different stiffnesses. Characteristic curves 3 shows the course of a characteristic curve in which the end effector gripping force is so high that the manipulated variable for the closing angle saturates at the maximum possible useful gripping force.

FIG. 17 shows, in contrast to FIG. 16, the characteristic curve under influence of different end effector gripping forces on the basis of the additive assessment of the relationship between gripping force and closing angle with the acting end effector gripping force. The characteristic curve 2 describes the intervention on a material which is stiffer in comparison to characteristic curve 1.

Preliminary tests show that a coupling of the gripping force and the kinematic component via a multiplication provides the better results and thus makes it easier for the user to differentiate between different material properties. Moreover, it is found that scaling factors and calculation methods can be selected depending on the nature of the environment of the end effector in order to get the optimal dynamic of the haptic perception for distinguishing special material parameters.

A necessary prerequisite for this method is the derivation of the interaction force F_{effector} between the gripping arms of the end effector (FIGS. 9-10). The dynamic requirements for these measurements are low, since the man's ability to exercise comprises only a small, almost quasi-static range. Therefore, the derivation of the force output values of the actuators and in the end effector is sufficient by integrating a sensor away from the end effector. This not only reduces the dynamic requirements but also the requirements for the space, weight and overload resistance of the sensor used.

The haptic feedback of the gripping force thus shown is quasi-static and can therefore be unsatisfactory when used for the representation of certain properties, such as the surface texture and the differentiation of materials and the like.

Therefore, in a further embodiment, this disadvantage is compensated in a simple manner by the integration of a highly dynamic actuator in the control means (piezo, voice coil, eccentric motor, etc.) with very small deflections required. Due to the properties of the human haptic perception, the introduction direction cannot be clearly distinguished in the case of highly dynamic signals, so that haptic feedback, which is felt in several degrees of freedom, can be represented with a one-dimensional movement of the actuator.

FIG. 22 shows a section as well as the exploded drawing of the embodiment of a control means with grip elements 1, actuators for tactile feedback 2, force sensor elements 3, a base of the control means 4 and a fastening element for TCP of the control means.

The measurement of the high-frequency signals could be carried out by measuring accelerations with miniaturized acceleration sensors arranged sterilisably in the end effector.

By comparing teleoperation systems with haptic feedback known from the literature, the invention not only expands the haptically perceptible range, but also reduces the design complexity of the entire control means. By using serially arranged actuators, a frequency distribution is possible for the haptic feedback. Instead of an actuator with a large bandwidth and, at the same time, great deflections in the base of the control means, the high-frequency portion of the haptic feedback is generated by a dynamic actuator with small deflections. In the end effector, the complexity of the sensor system is reduced so that multi-dimensional, highly dynamic force sensors can be replaced by a one-dimensional force sensor system and a multi-dimensional acceleration measurement. The latter is easier to integrate into the end effector since it does not have to be integrated into the main force flow direction. In addition, peripheral requirements for the sensors in terms of dynamics, overload resistance and sterilizable packaging are decreasing.

FIG. 23 shows one of two parallel kinematic mechanisms of the slave, which is also referred to as a manipulator in the following. Each manipulator has up to six degrees of freedom so that the TCP 1 can be positioned in the space. Furthermore, an end effector 2 attached to the TCP can be rotated about its longitudinal axis 3, angled (deviation) and its closing angle (Phi) can be changed.

The parallel kinematic mechanism consists of kinematic chains composed of rigid or flexible struts and joints. In general, a large number of solutions are conceivable for the implementation of the joints. Thus, in addition to rigid joints, solid body joints or flexible elements, e.g. Springs, film joints, folding bellows and NiTi wires could be used.

In order to move the intracorporal manipulator, six motors are preferably installed in the extra-corporal drive unit per manipulator. A different number of motors and gearboxes are conceivable. The movements generated are transmitted via three pushing rods 4 into the intracorporal region. Two active degrees of freedom are transmitted via a push rod in the form of translation q10-q30 and rotation q40-q60. The intracorporal movements are shaped by the parallel kinematic mechanism, which consists of a kinematic main chain 18 and up to four kinematic secondary chains 8, 9, 14, 15, such that a displacement of the push rods leads to a change in the position of the TCP, a rotation of the main chain Q40 rotates the end effector arbitrarily about its longitudinal axis, and a rotation q50 and q60 opens or closes the gripper. This is achieved e.g. by means of corresponding spindles which can also be seen in FIG. 23.

In detail, the parallel kinematic mechanism consists of a tripod-like substructure composed of the kinematic main chain 18 with four degrees of freedom and two kinematic secondary chains 8, 9 each with six degrees of freedom. These kinematic chains are connected to the main shaft 5 via pivot joints. In order to prevent jamming, these joints are realized as U-shaped clamping elements 6, 7. The rotation of the main chain is routed directly to the end effector via a universal joint located at the base so that the latter can be rotated freely about its longitudinal axis.

The rotations of the two remaining pushing rods are also transferred via cross joints along the first and second secondary chain, and are finally converted into a respective displacement via a spindle and a slide 10, 11. Via the third and fourth secondary chains 14, 15, each of which has four degrees of freedom, these movements are transmitted to sleds 21, 22 guided on the main shaft. In order to limit the forces occurring within the mechanism, compliances are integrated in the third and fourth secondary chain, in order to prevent jamming of the sled elements, these are also designed as a U-shaped bracket. The friction moment occurring within the rotary joints 12, 13 is dissipated via the secondary chains. For this purpose, the clamping elements 6 and 7 are connected to the elements 21 and 22 by means of a respective pendulum support.

Each of the displacements generated on the main shaft moves a push rod within the main shaft, this movement being applied to one of the two jaws of the end effector, e.g. by means of a cam disk or a toggle lever 16, 17. The pushing rods are guided by an elongated hole with respect to the main shaft and are locked against twisting by means of a pin. In order to obtain the rotation of the main shaft, the carriage movements 21, 22 produced on the main shaft are transmitted via pivot joints 12, 13 to the pushrod located in the shaft. As a result, the grippers can be opened or closed via a uniform rotation q50 and q60. If the push rods rotate counter-clockwise, the gripper is angled. The described state is invertible.

The parallel kinematic mechanism described has the following transfer characteristics:

1. The position of the TCP is independent of the rotations q40-q60 and is influenced by the shifts q10-q30 alone.
2. The push rods are arranged in a colinear manner so that the working space in the z direction is limited only by the maximum travel distance of the pushing rods. In the z-direction, a constant translation ratio of 1 results.
3. The longitudinal rotation of the end effector depends solely on the rotation q40.
4. The opening angle and the angle of inclination are mainly determined by the rotations q50 and q60.

In order to reference the kinematics with respect to the base plate (19), stops are attached (20) at the ends of the push rods.

The invention is not limited to the above-described embodiments but is intended to be defined by the claims.

1. A teleoperating system comprising:
a slave (10) which has a drive unit which drives a gripping end effector, wherein a kinematic coordinate of the end effector and a gripping force F_{effector} can be determined
with a camera (9) which is preferably integrated in the slave and which is aligned with the end effector,
a master (1) which is remote from the slave, having at least one operating unit (2, 3) on which a user can apply a gripping force F_{G}, the gripping force being transmitted to the slave, and a visual user interface 4), which represents the image of the camera,
where F_{G} is linearly dependent on the kinematic coordinate and the F_{effector}, or vice versa.

2. The teleoperation system of claim 1, wherein the F_{effector} is determined by one or more of the following approaches:
deduction of the force from the drive units of the drive unit in the slave or from a control computer;
measuring the current in the drive unit;
measuring the force in a kinematic structure between the end effector and the drive unit;
structure-integrated measurement by force sensors in parallel kinematics;
force/torque sensors on the drive unit;
measurement of the force directly between the end effector and the surrounding tissue.

3. The teleoperation system as claimed in claim 1, wherein the operating unit is as rigid as possible and has only the flexibility required for the gripping force detection.

4. The teleoperation system as claimed in claim 1, wherein the operating unit has a defined resilience and is thus designed for a defined deflection and thus enables gripping force detection, whereby an actuator in the operating unit can be dispensed with.

5. The teleoperation system as claimed in claim 1, wherein the gripping force F_{G} is determined by deriving the interaction force between the operating unit and the user by one or more of the following methods:
force measurement between the fingers
differential force measurement between the fingers
discharge of the force from the deflection or deformation of a non-rigid operating unit.

6. The teleoperation system as claimed in claim 1, wherein:
F_{G}=Kinematic coordinate*F_{effector}
Or
F_{G}=Kinematic coordinate+F_{effector}
or
F_{G}=Kinematic coordinate*(F_{effector}+Fₘᵢₙ)+F_{G}_{_}_{offset}
Where Fₘᵢₙ is the force to initially move the effector, and F_{G}_{_}_{offset} is the force to allow the sensor to respond in the operating unit, and preferably, possible factors for scaling the forces to adjust the described relationships to any of the manipulated environment conditions.

7. The teleoperation system as claimed in claim 1, characterized by a unit for generating tactile haptic feedback on the operating unit, wherein a frequency is transmitted by a sensor in the slave, which is sent to the unit for generating tactile haptic feedback Which is preferably in the range from about 50 to 1000 HZ.

8. The teleoperating system according to claim 7, wherein the tactile haptic feedback generating unit is one or more of the following:
force output by inertial mass motors
eccentric motors
piezoelectric actuators.

9. The teleoperating system as claimed according to claim 7, wherein an acting force direction of the tactile haptic feedback generating unit exerts no or only minimal forces in the direction of the gripping force F_{G}, in order to reduce control instability in the system.

10. The teleoperating system as claimed according to claim 7, wherein the frequency detected by a sensor in the slave is filtered as a function of ambient values in order to obtain stability in a control loop.

11. The teleoperating system as claimed according to claim 7, wherein the sensor in the slave is one or more of the following:
(SAW) sensors for detecting surface oscillations in the kinematic components or at the end effector.

12. The teleoperating system as claimed according to claim 1, wherein an additional digital representation of the current end effector coordinate can be superimposed in the camera image, preferably by one or more of the following:
angle indication, strokes which move towards each other, a stylized gripper that moves, color traces, representation of the force acting on the end effector on the display, deflection.

13. The teleoperating system as claimed according to claim 1, wherein a control computer is designed to carry out a differential force measurement on the operating unit by measuring the gripping force for the thumb and index finger separately from one another, and preferably the respective smaller or larger of the two measured values for The gripping force.

14. A slave for a teleoperation system, according to claim 1, comprising:
at least three tripods arranged as tripod, each having two active degrees of freedom in the form of translation and rotation, and each being driven by means of a drive into the degrees of freedom;
with an end effector which is connected to the push rods via kinematic chains, wherein the kinematic chains are designed in such a way that the end effector can be aligned and can be opened and closed in three dimensions by means of translation or rotation of the push rods.

15. The slave according to claim 14, wherein a kinematic chain is formed as a main chain, the rotation of which leads to a rotation of the end effector and the displacement thereof leads to a displacement of the end effector.

16. The slave according to claim 15, wherein two chains are formed as side chains, the displacement of which leads to a displacement of the end effector, and the rotation thereof leads to an opening or closing or bending.

17. The slave according to claim 16, wherein the rotations of the secondary chains are converted into a linear movement via a spindle and a carriage, which opens or closes the end effector.

18. The slave according to claim 14, wherein the kinematic main chain has at least four degrees of freedom and/or the kinematic secondary chain has at least six degrees of freedom.

19. The slave as claimed in claim 14, wherein the subchain is connected to the main chain by means of swivel joints, wherein the swivel joints are designed as U-shaped clamping elements.
